(19) United States
(12) Patent Application Publication
(10) Pub. No.: US 2015/0297282 A1
(43) Pub. Date: Oct. 22, 2015

(54) GENERATOR ASSEMBLIES FOR NEUROMODULATION THERAPY AND ASSOCIATED SYSTEMS AND METHODS

(71) Applicant: MEDTRONIC ARDIAN LUXEMBOURG SARL, Luxembourg (LU)

(72) Inventor: Hadar CADOURI, Santa Rosa, CA (US)

(21) Appl. No.: 14/379,822

(22) PCT Filed: Mar. 4, 2013
(86) PCT No.: PCT/US2013/028886

§ 371 Date: Aug. 20, 2014

Related U.S. Application Data
(60) Provisional application No. 61/606,456, filed on Mar. 4, 2012.
(60) Provisional application No. 61/636,581, filed on Apr. 20, 2012.

(51) Int. Cl. A61B 18/12; A61B 18/14

(52) U.S. Cl. CPC A61B 18/1206; A61B 18/1492; A61B 2018/00434

(57) ABSTRACT

Generator assemblies and systems for neuromodulation therapies are disclosed herein. A generator system configured in accordance with a particular embodiment of the present technology can include a stand assembly, a generator assembly carried by the stand assembly, and a display operably coupled to the generator assembly. The generator assembly can include at least one port configured to operably couple the generator assembly to a neuromodulation device such that the generator assembly can provide radio frequency (RF) energy to the neuromodulation device. The display can be configured to indicate operating conditions of the generator system during energy delivery. The generator system can further include a user interface operably coupled to the generator assembly and configured to activate and/or modulate the RF energy.

### TECHNICAL FIELD

The present technology relates generally to neuromodulation therapies. In particular, several embodiments are directed to generator assemblies for neuromodulation therapy and associated systems and methods.

### BACKGROUND

The sympathetic nervous system (SNS) is a primarily involuntary bodily control system typically associated with stress responses. Fibers of the SNS innervate tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (such as heart failure), and progressive renal disease. For example, radiotracer dilution has demonstrated increased renal norepinephrine (NE) spillover rates in patients with essential hypertension.

Cardio-renal sympathetic nerve hyperactivity can be particularly pronounced in patients with heart failure. For example, an exaggerated NE overflow from the heart and kidneys to plasma is often found in these patients. Heightened SNS activation commonly characterizes both chronic and end stage renal disease. In patients with end stage renal disease, NE plasma levels above the median have been demonstrated to be predictive for cardiovascular diseases and several causes of death. This is also true for patients suffering from diabetic or contrast nephropathy. Evidence suggests that sensory afferent signals originating from diseased kidneys are major contributors to initiating and sustaining elevated central sympathetic outflow.

Sympathetic nerves to the kidneys terminate in the blood vessels, the juxtaglomerular apparatus, and the renal tubules. Stimulation of the renal sympathetic nerves can cause increased renin release, increased sodium (Na⁺) reabsorption, and a reduction of renal blood flow. These neural regulation components of renal function are considerably stimulated in disease states characterized by heightened sympathetic tone and likely contribute to increased blood pressure in hypertensive patients. The reduction of renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and Water retention). These pharmacologic strategies, however, have significant limitations including limited efficacy, compliance issues, side effects, and others. Accordingly, there is a strong public-health need for alternative treatment strategics.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a neuromodulation system configured in accordance with an embodiment of the present technology.

FIG. 2 illustrates modulating renal nerves with an intravascular neuromodulation system in accordance with an embodiment of the present technology.

FIGS. 3A and 3B are front and back isometric views, respectively, of a generator system configured in accordance with an embodiment of the present technology.

FIG. 3C is an isometric view illustrating a generator console being detached from the generator system of FIGS. 3A and 3B in accordance with an embodiment of the present technology.

FIG. 3D is an isometric view of the generator console of FIG. 3C.

FIGS. 3E and 3F are isometric back views of the generator console of FIGS. 3C and 3D.

FIG. 3G is a perspective view of the generator system of FIGS. 3A-3F in a clinical environment in accordance with an embodiment of the present technology.

FIGS. 4A-4C are a series of isometric views of a generator system configured in accordance with another embodiment of the present technology.

FIG. 4D is an enlarged isometric view of a portion of the generator system of FIGS. 4A-4C.

FIG. 4E is an exploded isometric view of a generator assembly of the generator system of FIGS. 4A-4D configured in accordance with an embodiment of the present technology.

FIG. 4F is an isometric view of the generator assembly of FIG. 4E mounted to a support structure in accordance with an embodiment of the present technology.

FIG. 4G is a perspective view of the generator assembly of FIGS. 4E and 4F in a clinical environment in accordance with an embodiment of the present technology.

FIGS. 5A and 5B are isometric views of a generator system configured in accordance with yet another embodiment of the present technology.

FIG. 5C is a perspective view of the generator system of FIGS. 5A and 5B in a clinical environment in accordance with an embodiment of the technology.

FIG. 5D is a conceptual illustration of various configurations of the generator system of FIGS. 5A and 5B.

FIG. 6 is an isometric view of a generator system configured in accordance with a further embodiment of the present technology.

FIG. 7 illustrates various remote control devices for use with generator systems configured in accordance with embodiments of the present technology.

FIGS. 8A-8C are a series of screen shots illustrating a generator display configured in accordance with aspects of the present technology.

FIGS. 9A and 9B are screen shots illustrating a generator display configured in accordance with other aspects of the present technology.

FIGS. 10A-10D are a series of screen shots illustrating a generator display configured in accordance with further aspects of the present technology.

FIGS. 11A and 11B are screen shots of a display on a remote control device configured in accordance with aspects of the present technology.

FIG. 12 illustrates the integration of various displays of a generator system configured in accordance with an embodiment of the present technology.

FIG. 13 is a conceptual illustration of the sympathetic nervous system (SNS) and how the brain communicates with the body via the SNS.

FIG. 14 is an enlarged anatomic view of nerves innervating a left kidney to form the renal plexus surrounding the left renal artery.

FIGS. 15A and 15B are anatomic and conceptual views, respectively, of a human body depicting neural efferent and afferent communication between the brain and kidneys.

FIGS. 16A and 16B are anatomic views of the arterial vasculature and venous vasculature, respectively, of a human.

### DETAILED DESCRIPTION

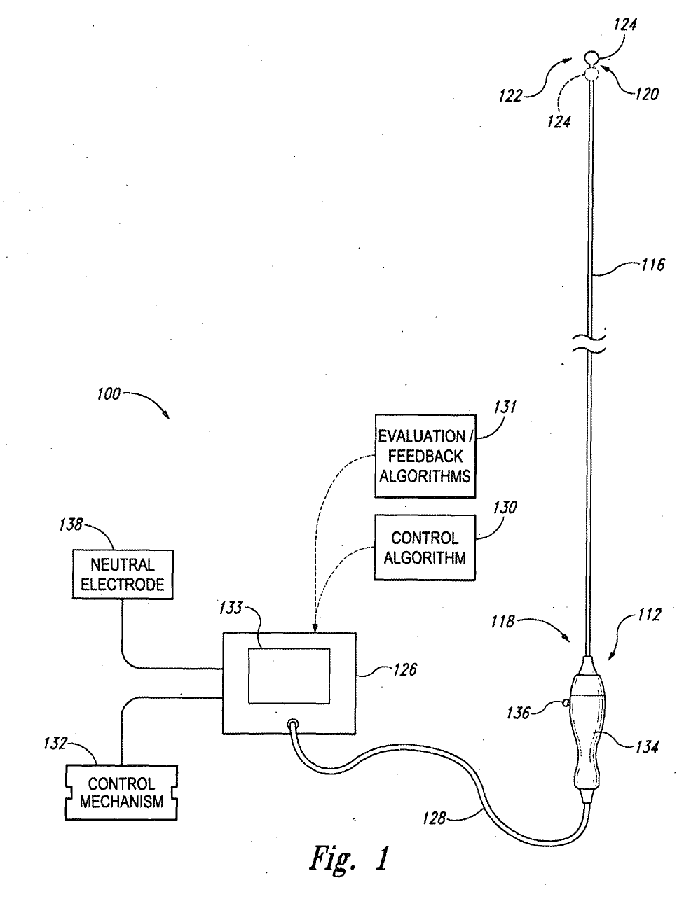

The present technology is generally directed to generator assemblies and systems for neuromodulation therapy (i.e., rendering neural fibers inert or inactive or otherwise completely or partially reduced in function). In various embodiments, generator assemblies and systems configured in accordance with the present disclosure can also provide feedback relating to operating conditions (e.g., temperature and impedance) during neuromodulation therapies. Specific details of several embodiments of the technology are described below with reference to FIGS. 1-16B. Although many of the embodiments are described below with respect to devices, systems, and methods for providing RF energy for renal neuromodulation, other applications (e.g., providing energy for intravascularly modulating other neural fibers) and other embodiments (e.g., using other forms of electrical energy and/or other types energy) in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-16B.

The terms “distal” and “proximal” are used in the following description with respect to a position or direction relative to the operator or the operator's control device (e.g., a handle assembly). “Distal” or “distally” arc a position distant from or in a direction away from the operator or the operator's control device. “Proximal” and “proximally” are a position near or in a direction toward the operator or the operator's control device.

### I. Renal Neuromodulation

Renal neuromodulation is the partial or complete incapacitation or other effective disruption of nerves innervating the kidneys. In particular, renal neuromodulation comprises inhibiting, reducing, and/or blocking neural communication along neural fibers (i.e., efferent and/or afferent nerve fibers) innervating the kidneys. Such incapacitation can be long-term (e.g., permanent or for periods of months, years, or decades) or short-term (e.g., for periods of minutes, hours, days, or weeks). Renal neuromodulation is expected to efficaciously treat several clinical conditions characterized by increased overall sympathetic activity, and in particular conditions associated with central sympathetic over stimulation such as hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, and sudden death. The reduction of afferent neural signals contributes to the systemic reduction of sympathetic tone/drive, and renal neuromodulation is expected to be useful in treating several conditions associated with systemic sympathetic over activity or hyperactivity. Renal neuromodulation can potentially benefit a variety of organs and bodily structures innervated by sympathetic nerves. For example, a reduction in central sympathetic drive may reduce insulin resistance that afflicts patients with metabolic syndrome and Type II diabetics. Additionally, osteoporosis can be sympathetically activated and might benefit from the downregulation of sympathetic drive that accompanies renal neuromodulation. A more detailed description of pertinent patient anatomy and physiology is provided in Section IV below.

Various techniques can be used to partially or completely incapacitate neural pathways, such as those innervating the kidney. The purposeful application of energy (e.g., electrical energy, thermal energy) to tissue by energy delivery element(s) can induce one or more desired thermal heating effects on localized regions of the renal artery and adjacent regions of the renal plexus (RP) (FIG. 14), which lay intimately within or adjacent to the adventitia of the renal artery. The purposeful application of the thermal heating effects can achieve neuromodulation along all or a portion of the renal plexus (RP).

The thermal heating effects can include both thermal ablation and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature can be above body temperature (e.g., approximately 37° C.) but less than about 45° C. for non-ablative thermal alteration, or the target temperature can be about 45° C. or higher for the ablative thermal alteration.

More specifically, exposure to thermal energy (heat) in excess of a body temperature of about 37° C., but below a temperature of about 45° C., may induce thermal alteration via moderate heating of the target neural fibers or of vascular structures that perfuse the target fibers. In cases where vascular structures are affected, the target neural fibers are denied perfusion resulting in necrosis of the neural tissue. For example, this may induce non-ablative thermal alteration in the fibers or structures. Exposure to heat above a temperature of about 45° C., or above about 60° C., may induce thermal alteration via substantial heating of the fibers or structures. For example, such higher temperatures may thermally ablate the target neural fibers or the vascular structures. In some patients, it may be desirable to achieve temperatures that thermally ablate the target neural fibers or the vascular structures, but that are less than about 90° C., or less than about 85° C., or less than about 80° C., and/or less than about 75° C. Regardless of the type of heat exposure utilized to induce the thermal neuromodulation, a reduction in renal sympathetic nerve activity (“RSNA”) is expected.

### II. Selected Embodiments of Renal Neuromodulation Systems

FIG. 1 illustrates a neuromodulation system 100 (“system 100”) configured in accordance with an embodiment of the present technology. The system 100 includes a treatment device 112 operably coupled to an energy source or energy generator 126 (e.g., an RF energy generator). In the embodiment shown in FIG. 1, the treatment device 112 (e.g., a catheter) includes an elongated shaft 116 having a proximal portion 118, a handle assembly 134 at a proximal region of the proximal portion 118, and a distal portion 120 extending distally relative to the proximal portion 118. The treatment device 112 further includes a treatment section or therapeutic assembly 122 including an energy delivery element 124 (e.g., an electrode) at or near the distal portion 120 of the shaft 116. In the illustrated embodiment, a second energy delivery element 124 is illustrated in broken lines to indicate that the systems and methods disclosed herein can be used with treatment devices having one or more energy delivery elements 124. Further, it will be appreciated that although only two energy delivery elements 124 are shown, the treatment device 112 may include additional energy delivery elements 124 (e.g., four electrodes). For example, the therapeutic assembly 122 can be configured to have a helical shape with a plurality of energy delivery elements 124 positioned thereon. Other helical and/or multi-electrode therapeutic assemblies 122 may have energy delivery elements 124 with different positions relative to one another than those shown in FIG. 1.

The therapeutic assembly 122 can be delivered intravascularly to a treatment site (e.g., a renal artery) in a low-profile configuration. In one embodiment, for example, the distal end of the therapeutic assembly 122 may define a passageway for engaging a guide wire (not shown) for delivery of the treatment device 112 using over-the-wire (“OTW”) or rapid exchange (“RX”) techniques. At the treatment site, the therapeutic assembly 122 can transform to a deployed state or arrangement for delivering energy at the treatment site and providing therapeutically-effective electrically-induced and/or thermally-induced renal neuromodulation. In various embodiments, the therapeutic assembly 122 may be placed or transformed into the deployed state via remote actuation using an actuator 136, such as a knob, pin, or lever carried by the handle assembly 134. In other embodiments, however, the therapeutic assembly 122 may be transformed between the delivery and deployed states using other suitable mechanisms or techniques. In some embodiments, for example, the therapeutic assembly 122 may be delivered to the treatment site within a guide sheath (not shown). When the therapeutic assembly 122 is at the target site, the guide sheath may be at least partially withdrawn or retracted and the therapeutic assembly 122 can move from the low-profile state to the deployed arrangement.

The energy generator 126 (e.g., an RF energy generator) is configured to generate a selected form and magnitude of energy for delivery to the treatment site via the energy delivery element(s) 124. The energy generator 126 can be electrically coupled to the treatment device 112 via a cable 128. At least one supply wire (not shown) can pass along the elongated shaft 116 or through a lumen in the elongated shaft 116 to the energy delivery element(s) 124 and transmits the treatment energy to the energy delivery element(s) 124. As described in greater detail below, a control mechanism, such as a foot pedal 132 or a handheld controller, may be connected (e.g., pneumatically connected or electrically connected) to the energy generator 126 to allow the operator to initiate, terminate and, optionally, adjust various operational characteristics of the energy generator 126 (e.g., power delivery).

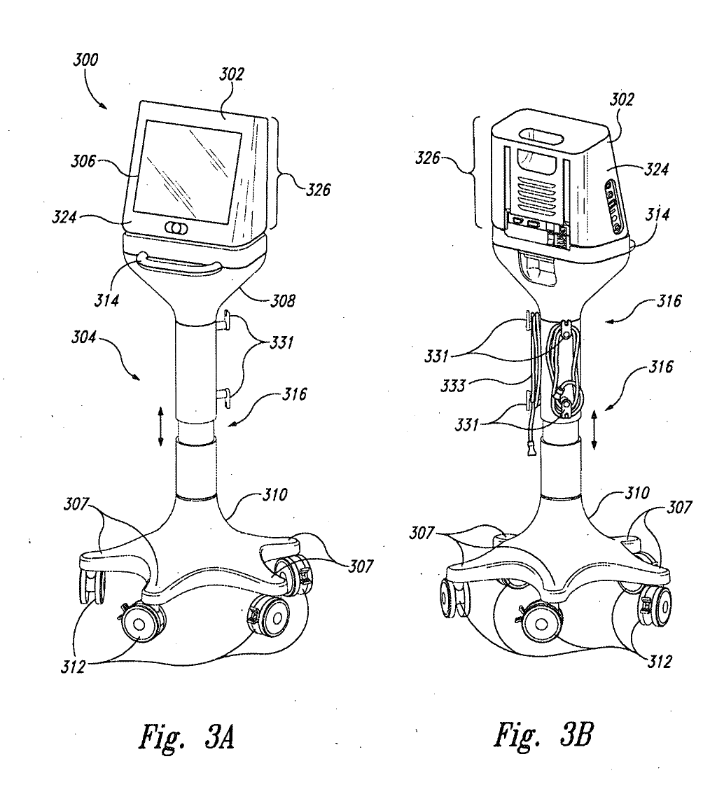

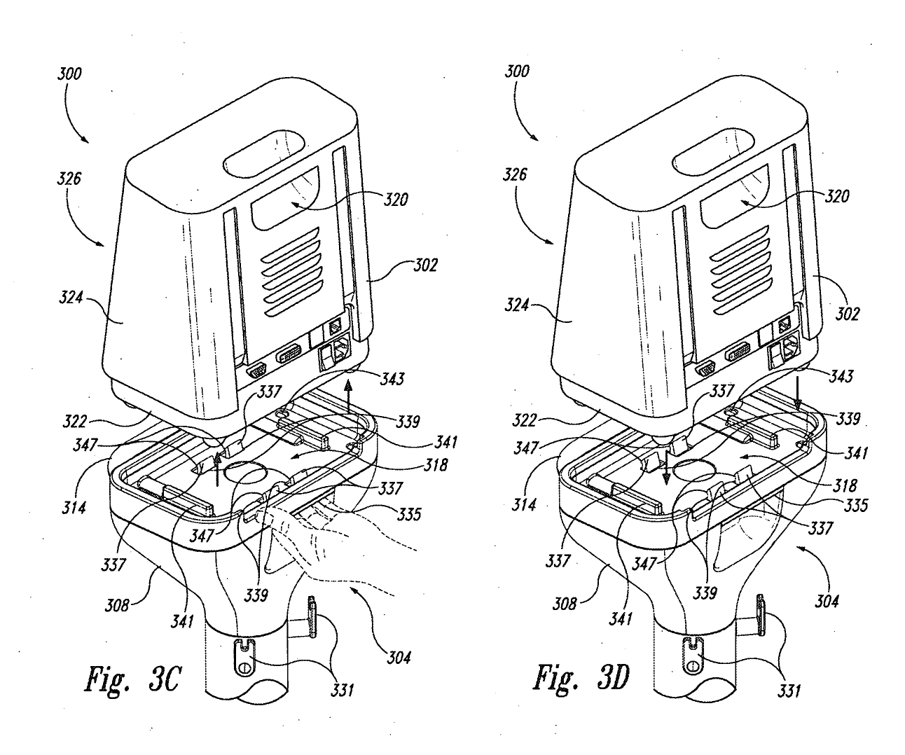

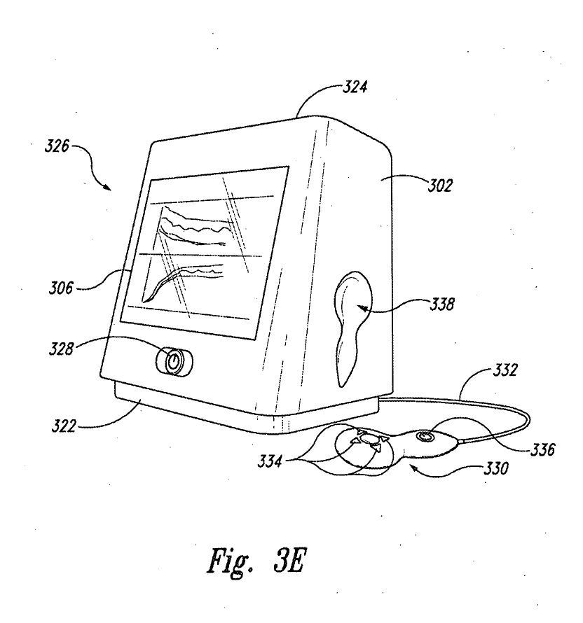

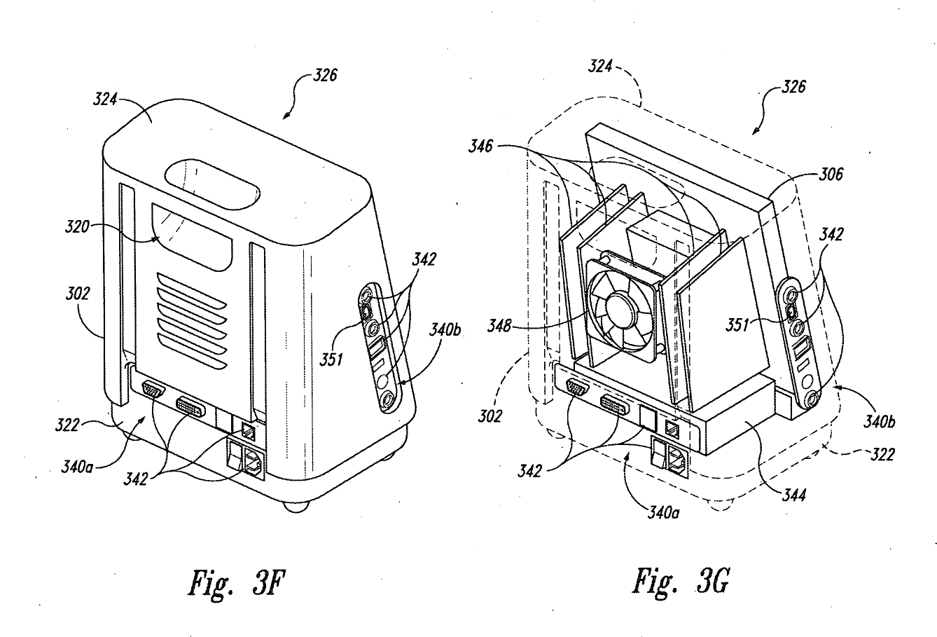

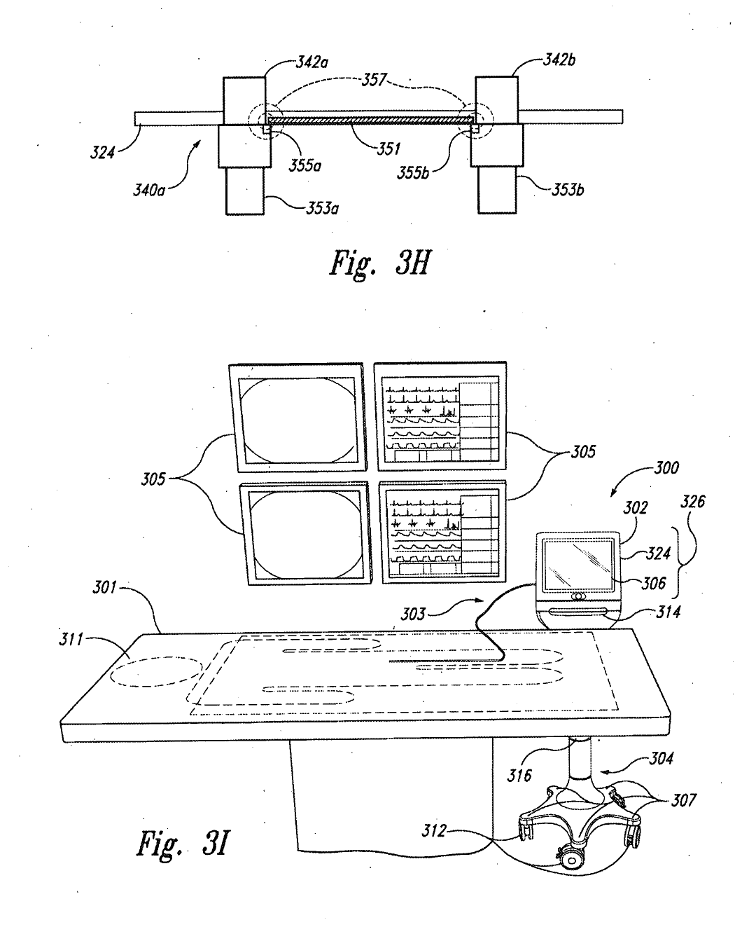

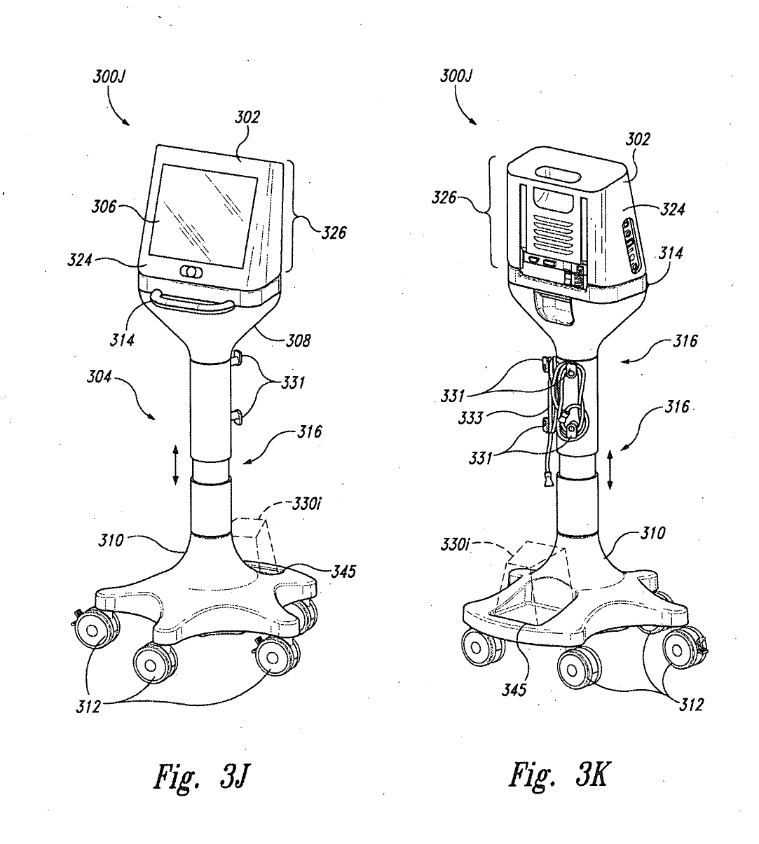

The energy generator 126 can be configured to deliver the treatment energy via an automated control algorithm 130 and/or under the control of a clinician. For example, the energy generator 126 can include computing devices (e.g., personal computers, server computers, tablets, etc.) having processing circuitry (e.g., a microprocessor) that is configured to execute stored instructions relating to the control algorithm 130. In addition, the processing circuitry may be configured to execute one or more evaluation/feedback algorithms 131 and may provide feedback to the user (e.g., via a display 133). The display 133 and/or associated features may be configured to provide indications of power levels or sensor data, such as audio, visual and/or other indications, or may be configured to communicate the information to another device. For example, the energy generator 126 may be remotely coupled to a monitor in a catheterization laboratory. Further details regarding suitable feedback displays, control devices, and associated energy generators are described below with reference to FIGS. 3A-12.

The computing devices associated with the system 100 can further include memory devices, input devices (e.g., a keyboard, mouse, touchscreen, etc.), output devices (e.g., a display device), and storage devices (e.g., disk drives). The output devices may be configured to communicate with the treatment device 112 (e.g., via the cable 128) to control power to the energy delivery element(s) 124 and/or to obtain signals from the energy delivery element(s) 124 or any associated sensors (not shown). The memory and storage devices are computer-readable media that may be encoded with computer-executable instructions that implement the control algorithm 130 and/or evaluation/feedback algorithm(s) 131. The instructions, data structures, and message structures may be stored or transmitted via a data transmission medium, such as a signal on a communications link (e.g., the Internet, a local area network, a wide area network, a point-to-point dial-up connection, a cell phone network, etc.).

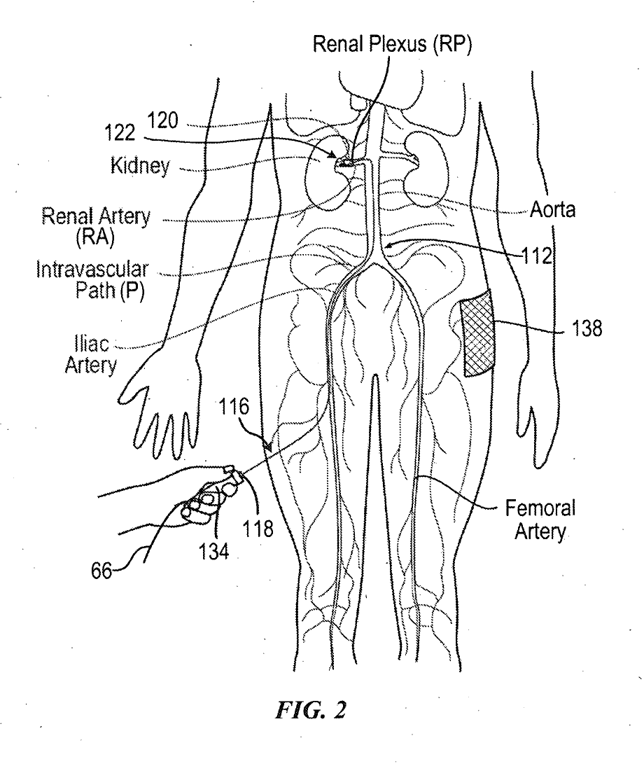

In selected embodiments, the system 100 may be configured to provide delivery of a monopolar electric field via the energy delivery element 124. In such embodiments, a neutral or dispersive electrode 138 may be electrically connected to the energy generator 126 and attached to the exterior of the patient (FIG. 2). Additionally, one or more sensors (not shown), such as one or more temperature (e.g., thermocouple, thermistor, etc.), impedance, pressure, optical, flow, chemical and/or other sensors, may be located proximate to or within the energy delivery element 124. The sensor(s) and the energy delivery element 124 can be connected to one or more supply wires (not shown) that transmit signals from the sensor(s) and/or convey energy to the energy delivery element(s) 124.

In embodiments including multiple energy delivery elements 124, the energy delivery elements 124 may deliver power independently (i.e., may be used in a monopolar fashion), either simultaneously, selectively, or sequentially, and/or may deliver power between any desired combination of the energy delivery elements 124 (i.e., may be used in a bipolar fashion). Furthermore, the operator optionally may be permitted to choose which energy delivery element(s) 124 are used for power delivery in order to form highly customized lesion(s) within the renal artery, as desired.

FIG. 2 illustrates modulating renal nerves with an embodiment of the system 100 of

FIG. 1. The treatment device 112 provides access to the renal plexus through an intravascular path, such as from a percutaneous access site in the femoral (illustrated), brachial, radial, or axillary artery to a targeted treatment site within a respective renal artery. As illustrated, a section of the proximal portion 118 of the shaft 116 is exposed externally of the patient. By manipulating the proximal portion 118 of the shaft 116 from outside the intravascular path (e.g., via the handle assembly 134), the operator may advance the shaft 116 through the sometimes tortuous intravascular path and remotely manipulate or actuate the distal portion 120 of the shaft 116. Image guidance, e.g., computed tomography (CT), fluoroscopy, intravascular ultrasound (IVUS), optical coherence tomography (OCT), or another suitable guidance modality, or combinations thereof, may be used to aid the operator's manipulation. Further, in some embodiments, image guidance components (e.g., IVUS, OCT) may be incorporated into the treatment device 112 itself.

After the therapeutic assembly 122 is adequately positioned in the renal artery), it can be deployed (e.g., radially expanded) and manipulated using the handle 134 or other suitable means until the therapeutic assembly 122 (e.g., the energy delivery element 124) is positioned at its target site in stable contact with the inner wall of the renal artery. The purposeful application of energy from the energy generator 126 (FIG. 1) to tissue of the renal artery by the energy delivery element 124 can induce one or more desired neuromodulating effects on localized regions of the renal artery and adjacent regions of the renal plexus, which lay intimately within, adjacent to, or in close proximity to the adventitia of the renal artery). This purposeful application of the energy may achieve neuromodulation along all or at least a portion of the renal plexus.

The neuromodulating effects are generally a function of, at least in part, power, time, contact between the energy delivery elements 124 and the vessel wall, and blood flow through the vessel. The neuromodulating effects may include denervation, thermal ablation, and non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.). but less than about 45° C. for non-ablative thermal alteration, or the target temperature may be about 45° C. or higher for the ablative thermal alteration. Desired non-thermal neuromodulation effects may include altering the electrical signals transmitted in a nerve.

### III. Generator Assemblies and Systems

With the foregoing discussion of neuromodulation systems in mind, a variety of different generator systems and related components for use with such neuromodulation systems are described below with reference to FIGS. 3A-12. The generator systems and related components can serve as an energy source (e.g., the generator 126 of FIG. 1) for the neuromodulation systems and provide feedback related to the treatment sessions. It will be appreciated that the generator systems described below and/or specific features thereof can be used with the neuromodulation system components described above (e.g., the treatment device 112 shown in FIGS. 1 and 2), used with other suitable neuromodulation system components, and/or used as standalone or self-contained devices.

FIGS. 3A and 3B are front and back isometric views, respectively, of a generator system 300 (“system 300”) configured in accordance with an embodiment of the present technology. The system 300 can include an energy source or generator assembly 302 and a display 306 carried by a base, cart, or stand assembly 304. The generator assembly 302 can be configured to convey energy to a neuromodulation treatment device (e.g., the treatment device 112 shown in FIG. 1). In various embodiments, for example, the generator assembly 302 can provide one or more channels of RF energy to a single or multi-electrode treatment device. The display 306 can be configured to communicate information related to neuromodulation therapies, such as the operating conditions (e.g., impedance, temperature) of the treatment device and/or the generator assembly 302.

The stand assembly 304 can include a first or body portion 308 configured to carry the generator assembly 302 and the display 306 and a second or base portion 310 configured to provide a stable base structure for the system 300. In some embodiments, the base portion 310 can include one or more wheels 312 to facilitate transportation of the system 300, and the body portion 308 can include a grip or handle 314 to assist with transportation. In various embodiments, the stand assembly 304 can include an adjustable member 316 that allows users to change the height of the system 300 (e.g., by approximately 30 cm). As such, the system 300 can be raised (e.g., to accommodate standing use) and lowered (e.g., to accommodate seated use and/or storage). The stand assembly 304 can also be configured to store cords (not shown) associated with the generator assembly 302 and/or the display 306 (e.g., power cords, video connector cables, etc.) in the body portion 308 and/or base portion 310. In other embodiments, the stand assembly 304 may have a different arrangement and/or different features.

The display 306 can include a screen or monitor that has a suitable resolution and size to illustrate various operating conditions of the system 300 and/or other related information. For example, the display 306 can be approximately 9-12 inches (22.9-30.5 cm) and include a digital visual interface (“DVI”), a visual graphics array (“VGA”), a high-definition multimedia interface (“HDMI”), and/or other high resolution displays. In other embodiments, the display 306 can be larger or smaller and/or be coupled to other types of displays or indicators (e.g., audible indicators, LED indicators, etc.) The display 306 can also be configured as a touchscreen that serves as a user interface for controlling the system 300 and/or otherwise interacting with the operator.

In the embodiment illustrated in FIGS. 3A and 3B; the generator assembly 302 and the display 306 are integrated into a single housing 324 such that they form a generator console 326 (“console 326”). The console 326 can serve as a compact standalone energy source that provides RF or other forms of energy via the generator assembly 302 and communicates operating conditions of neuromodulation therapies via the display 306. FIG. 3C, for example, illustrates the console 326 as it is being separated from the stand assembly 304. In the illustrated embodiment, the stand assembly 304 includes a recessed portion 318 configured to receive a corresponding protruding portion, e.g., a pedestal portion 322, of the generator assembly 302. The recessed portion 318 and the pedestal portion 322 can form an interference fit when mated together such that the console 326 is removably mounted to the stand assembly 304. A grip or handle portion 320 on the console 326 can be used to pull the console 326 apart from the stand assembly 304 and facilitate subsequent transport of the console 326. In other embodiments, the console 326 can be removably attached to the stand assembly 304 using other suitable fastening methods known in the art.

The portable console 326 has a relatively small footprint, and can therefore be positioned virtually anywhere in a catheterization laboratory or other suitable location for neuromodulation therapy. For example, the console 326 can rest on an equipment bench proximate a patient table or on the patient table itself. The console 326 may also be mounted to another structure, such as a bed rail or an I.V. pole (e.g., using a VESA mount). In other embodiments, however, the display 306 and the generator assembly 302 can be separate components that are spaced apart from one another. For example, the generator assembly 302 and the display 306 can be positioned on different portions of the stand assembly 304, or the display 306 may be positioned in a remote location (e.g., proximate to other monitors in a catheterization laboratory and/or integrated with the monitors in the catheterization laboratory) and operably coupled to the generator assembly 302. Further details regarding such arrangements are described below.

FIGS. 3D-3F illustrate various isometric views of the console 326. Referring first FIG. 3D, the console 326 can include a power button 328 configured to activate the generator assembly 302 or the console 326 as a whole and a user interface configured to control the application of energy to a treatment site (e.g., an inner wall of a renal artery) via a neuromodulation device. In the illustrated embodiment, the user interface includes a durable (i.e., non-disposable) remote control device 330 that is operatively coupled to the generator assembly 302 via a cable 332. In other embodiments, the remote control device 330 can be disposable and/or wirelessly coupled to the generator assembly 302. The remote control device 330 can include an energy activation button 336 that initiates delivery of RF or other forms of energy to the treatment site and a plurality of command buttons 334 used to modulate energy delivery. As shown in FIG. 3D, the command buttons 334 can be arranged in a circular pattern that distinguishes the buttons 334 from one another such that the operator can control energy delivery without having to look down at the remote control device 330. In other embodiments, the remote control device 330 can include additional command buttons, some of the buttons 334, 336 can be omitted, and/or the buttons 334, 336 can have other suitable configurations. In various embodiments, the console 326 can be configured to retain the remote control device 330 for compact storage. For example, in the embodiment illustrated in FIG. 3D, the console 326 includes a recessed portion 338 of the housing 324 configured to receive the remote control device 330.

As shown in FIG. 3E, the console 326 can further include a first cable interface 340a associated with the generator assembly 302 and a second cable interface 340b associated with the display 306 (FIG. 3D). The first and second cable interfaces 340a, 340b (referred to collectively as the cable interfaces 340) can include one or more adaptors or ports 342 configured connect the generator assembly 302, the display 306 (FIG. 3D), and/or other features of the console 326 with various peripheral devices. For example, the first cable interface 340a can include a port for a neuromodulation device, a port for a return electrode (e.g., configured to convey sensed data from the neuromodulation device), a port for a neutral or dispersive electrode, a USB port, an AC power supply port, a network connection port, and/or other suitable ports. The second cable interface 340b can include various video and audio ports related to the display 306. In the illustrated embodiment, the first cable interface 340a is exposed through an opening in the housing 324 at the back of the console 326, and the second cable interface 340b is exposed through an opening at the side of the console 326. In other embodiments, however, the cable interfaces 340 can be located elsewhere on the console 326, the cable interfaces 340 can be combined into a single cable interface, and/or the console 326 can include additional cable interface. In further embodiments, the two cable interfaces 340 can include identical ports 342, therefore allowing the operator to select which location of the cable interfaces 340 (e.g., the back or the side) is more convenient to use.

FIG. 3F illustrates selected internal features of the console 326, such as the generator assembly 302. In the illustrated embodiment, the generator assembly 302 includes a power supply 344, a fan 348, and four RF boards 346 configured to convey RF energy through four channels that correspond with one or more electrodes of a neuromodulation device. In other embodiments, the generator assembly 302 can include one, two, three, or more than four RF boards 346 and/or be configured to generate other forms of energy. In further embodiments, the console 326 can include additional features that facilitate energy generation and/or provide monitoring and feedback related to energy delivery.

FIG. 3G is a perspective view of the system 300 in a clinical environment (e.g., a catheterization laboratory) configured in accordance with an embodiment of the present technology. The system 300 has a relatively small footprint, and can therefore be positioned in various locations in the clinical setting, such as proximate to a patient (not shown). For example, in the illustrated embodiment, the system 300 is positioned behind a patient table 301 and the display 306 is substantially aligned with other screens or monitors 305 (e.g., fluoroscopy screens for image guidance) by manipulating the adjustable member 316. In other embodiments, the console 326 can be disconnected from the stand assembly 304 and configured to rest on the patient table 301 or be mounted to a nearby structure (e.g., an I.V. pole or bed rail), thereby further minimizing the space required for the console 326 in the clinical environment.

During a neuromodulation procedure, locations near the patient are typically within a sterile field. Accordingly, the console 326 and/or the system 300 (depending upon the manner in which it is used) can be configured for use within the sterile field. Rather than sterilizing the system 300, a sterile barrier (e.g., a bag) can be provided around all or a portion of the system 300. In the embodiment illustrated in FIG. 3G, the console 326 is positioned outside the sterile field. A neuromodulation device 303 is coupled to the generator assembly 302 (e.g., via the first connection interface 340a shown in FIGS. 3E and 3F) such that it extends from the console 326 into the sterile field where it can be navigated through the vasculature of a patient to a target site (e.g., a renal artery). Once at the target site, an operator can convey energy (e.g., RF energy) to the distal portion of the neuromodulation device and control the application thereof using the remote control device 330 (FIG. 3D), which may also be covered in a separate bag to maintain the sterile field. In other embodiments, the system 300 can include other user interfaces (e.g., a touchscreen, foot pedal, etc.) for controlling energy delivery.

As mentioned previously, the display 306 can be configured to show operating characteristics of neuromodulation device 303 and the generator assembly 302 before, during, and/or after energy delivery. For example, the display 306 can be configured to plot the impedance of one or more electrodes at the distal portion of the neuromodulation device in real time to assist the operator in determining whether sufficient contact has been made between the electrodes and the target site. Such impedance plots can also be used to indicate the status of each electrode (e.g., whether the electrode has moved, if the treatment is working properly, etc.). For example, a change in impedance can indicate an instability in the electrode's contact with the tissue. The display 306 may also or alternatively be configured to indicate the temperature at each electrode, which can be used to identify, e.g., constrictions in the vessel or the pulsation of the blood. The display 306 can also include other information related to neuromodulation therapies. In various embodiments, the system 300 can be coupled to the remote monitors 305 to selectively use these additional screens to view the operational characteristics of the system 300, e.g., to view additional information or to display the same information as display 306, but on a larger screen. Upon completion of the neuromodulation therapy, the system 300 can be placed in a compact configuration (e.g., by changing the height via the adjustable member 316) for convenient storage, or the console 326 can be stored separate from the stand assembly 304 on a shelf or in a cabinet.

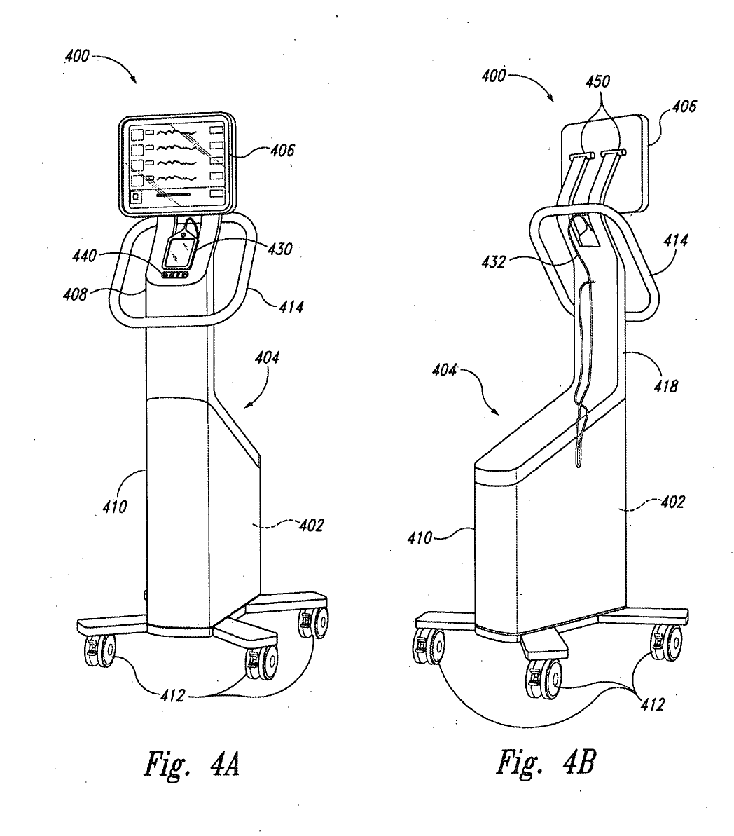

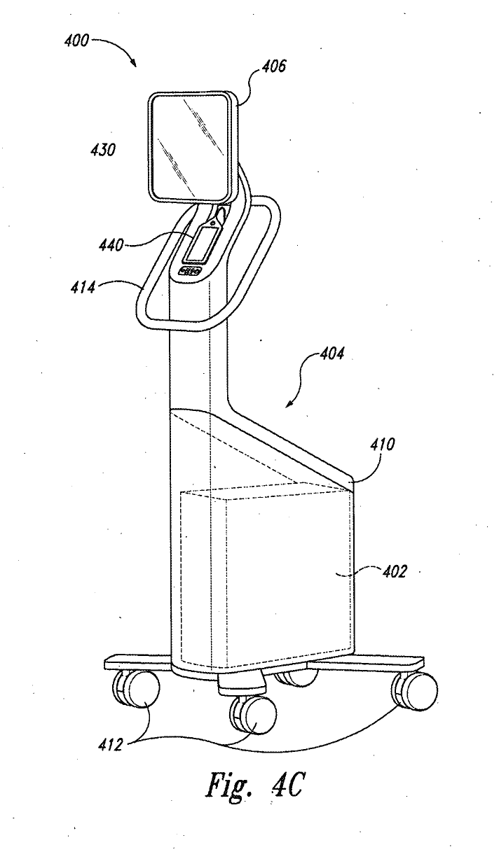

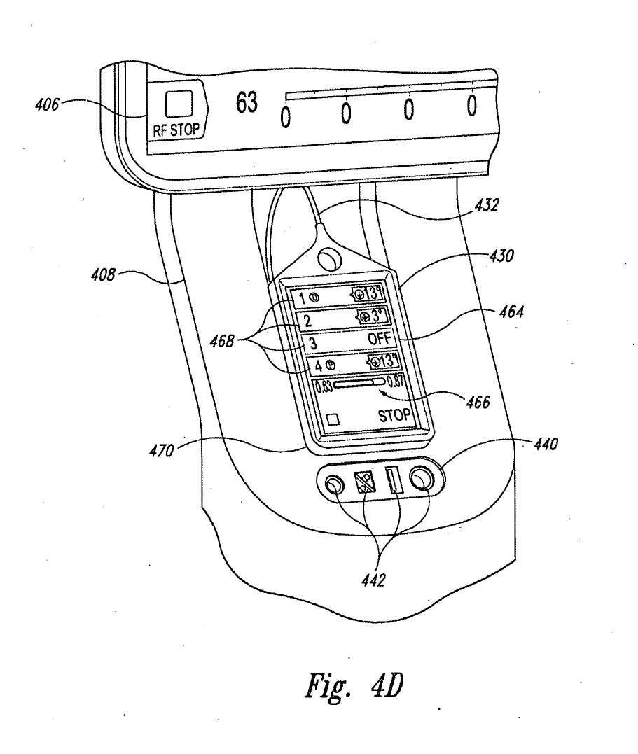

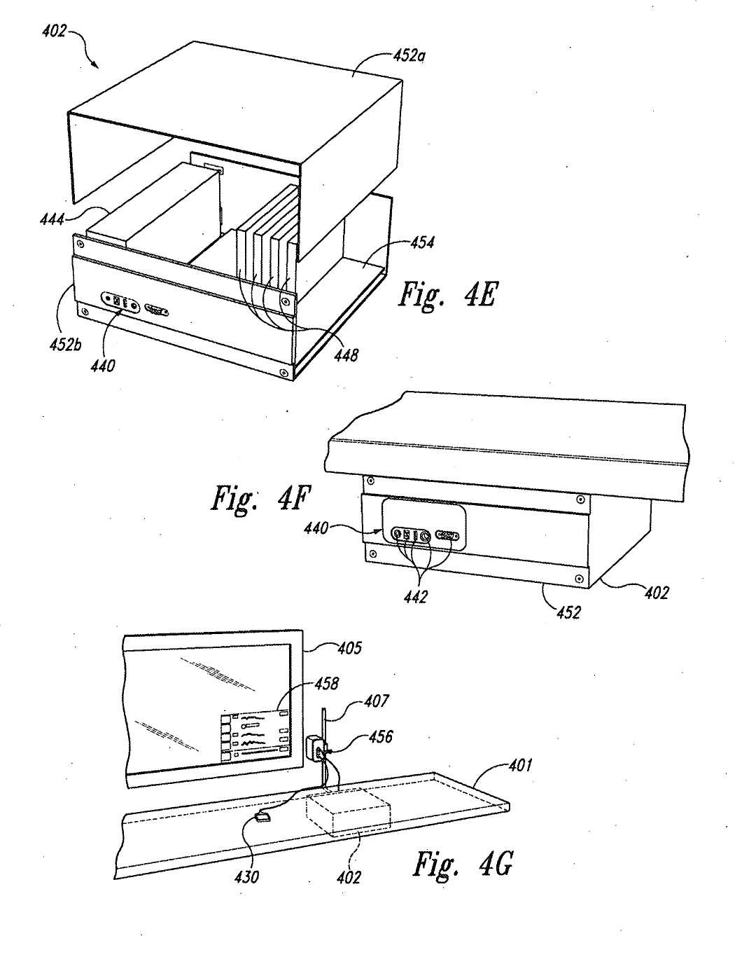

FIGS. 4A-4C are isometric views of a generator system 400 (“system 400”) configured in accordance with another embodiment of the present technology. The system 400 can include features generally similar to the system 300 described above with reference to FIGS. 3A-3G. For example, the system 400 can include a generator assembly 402 (e.g., an RF generator) and a display 406 carried by a stand assembly 404. In this embodiment, however, the generator assembly 402 and the display 406 are not integrated into a single console, but are separate components spaced apart from one another on the, stand assembly 404. In the illustrated embodiment, for example, the generator assembly 402 is housed in a base portion 410 of the stand assembly 404 (e.g., in a sheet metal shroud; FIG. 4C), and the display 406 is mounted on supports 450 (FIG. 4B) extending from a body portion 408 of the stand assembly 404. The generator assembly 402 and the display 406 can be operably coupled together using a wireless connection or electrical connectors. (not shown) extending through the body of the stand assembly 404. In various embodiments, the supports 450 can be configured to swivel, tilt, and/or adjust the height of the display 406 to change the viewing angle of the display 406 (e.g., for operators of different heights) and/or reduce glare. A handle 414 can extend around the body portion 408 of the stand assembly 404 to provide a barrier around the system 400 that reduces the likelihood of damage to the display 406, and also aids in maneuvering the stand assembly 404.

As shown in FIGS. 4A and 4C, a cable interface 440 can be spaced apart from and operably coupled to the generator assembly 402 such that the cable interface 440 is positioned at an easily accessible location on the body portion 408 of the stand assembly 404. FIG. 4D is an enlarged view of the cable interface 440 on the body portion 408 of the stand assembly 404. The ports 442 can be configured to connect the generator assembly 402 with, e.g., a neuromodulation device, a return electrode, and/or other suitable peripheral devices. In other embodiments, the system 400 can include additional cable interfaces located elsewhere on the system 400. For example, a cable interface on the generator assembly 402 may be accessed via an opening in the housing of the base portion 410.

As further shown in FIG. 4D, the body portion 408 of the stand assembly 404 can also include a holder or recess 470 configured to retain a remote control device 430. Similar to the remote control device 330 described above with reference to FIG. 3D, the remote control device 430 in the illustrated embodiment is a durable handheld device that is hardwired to the generator device 402 via a cable 432. However, rather than physical command buttons, the remote control device 430 includes a touchscreen 464 that receives commands (e.g., to control energy delivery) via finger taps. As will be described in greater detail below, the touchscreen 464 can also be configured to provide visual indicators related to the operating conditions of the system 400 and the neuromodulation device. For example, in the illustrated embodiment, the touchscreen 464 includes a timer 466 and a plurality of RF channel indicators 468 that provide operational characteristics (e.g., temperature, impedance, activation status, etc.) corresponding to one or more electrodes on a neuromodulation device. In other embodiments, the touchscreen 464 can display other information related to the system 400. In further embodiments, the remote control device 430 can also include physical control buttons to supplement the touchscreen 464.

Referring back to FIGS. 4C and 4D together, the system 400 can be rolled to a desired location within a clinical environment using a plurality of wheels 412 at the base portion 410 of the stand assembly 404, which can be locked to secure the system 400 in place. A neuromodulation device and other peripheral devices can be operatively coupled to the system 400 via the easily accessible cable interface 440. Once the distal portion of the neuromodulation device is positioned at a treatment site (e.g., a renal artery), the remote control device 430 can be used to initiate and control the application of energy (e.g., RF energy) from the generator assembly 402 to the treatment site. The remote control device 430 can remain mounted on the holder 470 during the treatment for easy viewing, or the remote control device 430 can be removed from the holder 470, (e.g., enclosed in a sterile barrier for use by the treating clinician). In various embodiments, the display 406 can be configured as a touchscreen to serve as an additional or alternative control mechanism. For example, the display 406 may be used to control aspects of the system 400 that benefit from a larger visual display. Before, during, and/or after the application of energy to the treatment site, the display 406 and/or the touchscreen 464 on the remote control device 430 can provide real time information related to the operating conditions of the system 400 and the neuromodulation device. Once the procedure is complete, the system 400 can conveniently be rolled into a storage space for future use.

In various embodiments, the system 400 can be configured to allow the generator assembly 402 to be used as a standalone device independent of the stand assembly 404. For example, FIGS. 4E and 4F illustrate the generator assembly 402 of FIGS. 4A-4C after being removed from the stand assembly 404. The generator assembly 402 can include features generally similar to the features of the generator assembly 302 described above with reference to FIGS. 3A-3G. As shown in FIG. 4E, for example, the generator assembly 402 includes a power supply 444 and a plurality of RF daughter boards 448 coupled to a printed circuit board assembly (“PCBA”) 454. A housing 452 (shown in FIG. 4E as a first housing portion 452a and a second housing portion 452b) can be made from a durable material, such as a sheet metal or milled aluminum, to enclose and protect the internal components of the generator assembly 402. A cable interface 440 can project through the housing 452 to provide access to ports 442 (FIG. 4F) that can be used to couple the generator assembly to other devices (e.g., a neuromodulation device, USB, return electrode, monitor, remote control device, etc.). The generator assembly 402 can also include a power plug and/or additional connection ports positioned elsewhere on the housing 452.

The generator assembly 402 can be permanently or semi-permanently mounted to a support structure, such as a patient table, in an equipment rack, and/or on another suitable support structure. FIG. 4G, for example, is a perspective view of the generator assembly 402 mounted to an underside of a patient table 401 in accordance with an embodiment of the present technology. In the illustrated embodiment, the generator assembly 402 is operably coupled to a cable interface module 456 configured to connect the generator assembly 402 with a neuromodulation device (not shown), the remote control device 430, a remote display 405, and/or other peripheral devices. The cable interface module 456 can be positioned in an easily accessible location, such as on a pole 407 proximate the patient table 401 and may include a convenient place to store the remote control device 430. In other embodiments, peripheral devices can be coupled directly to the generator assembly 402 via the cable interface 440 (FIG. 4F).

The remote display 405 can serve as the display for the system 400 and can display the operating conditions of the generator assembly 402. In the embodiment illustrated in FIG. 4G, for example, the display 405 includes a picture-in-picture (PIP) inset 458 on which the operating conditions are displayed, leaving the remainder of the display 405 free to show other information (e.g., fluoroscopic image guidance displays). In other embodiments, the generator assembly 402 can be coupled to a dedicated display and/or the remote control device 430 that can also display operational conditions to supplement the information shown on the display 405.

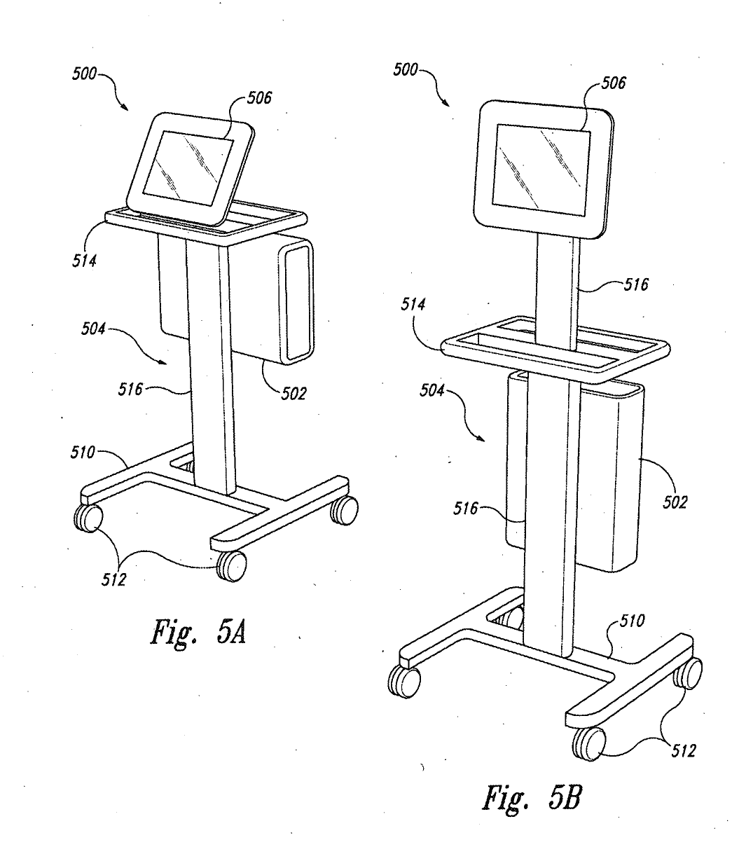

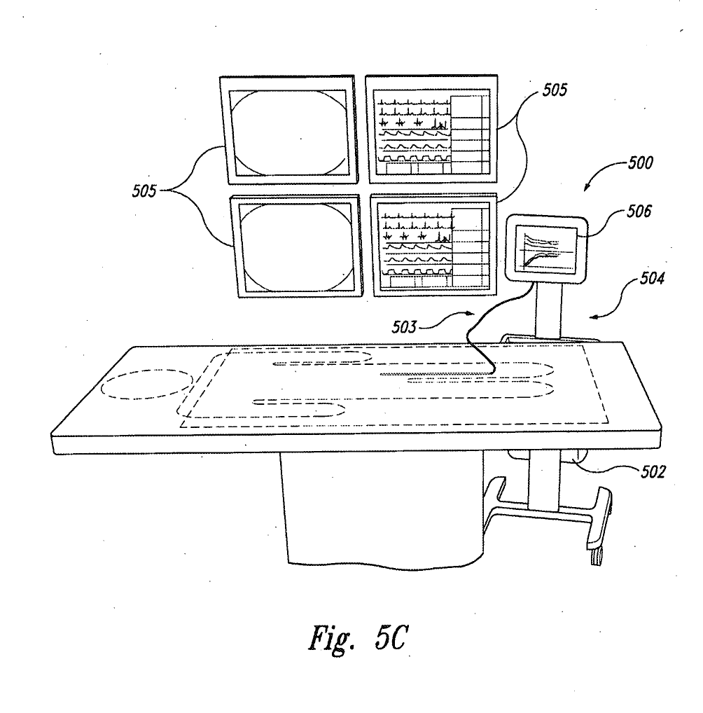

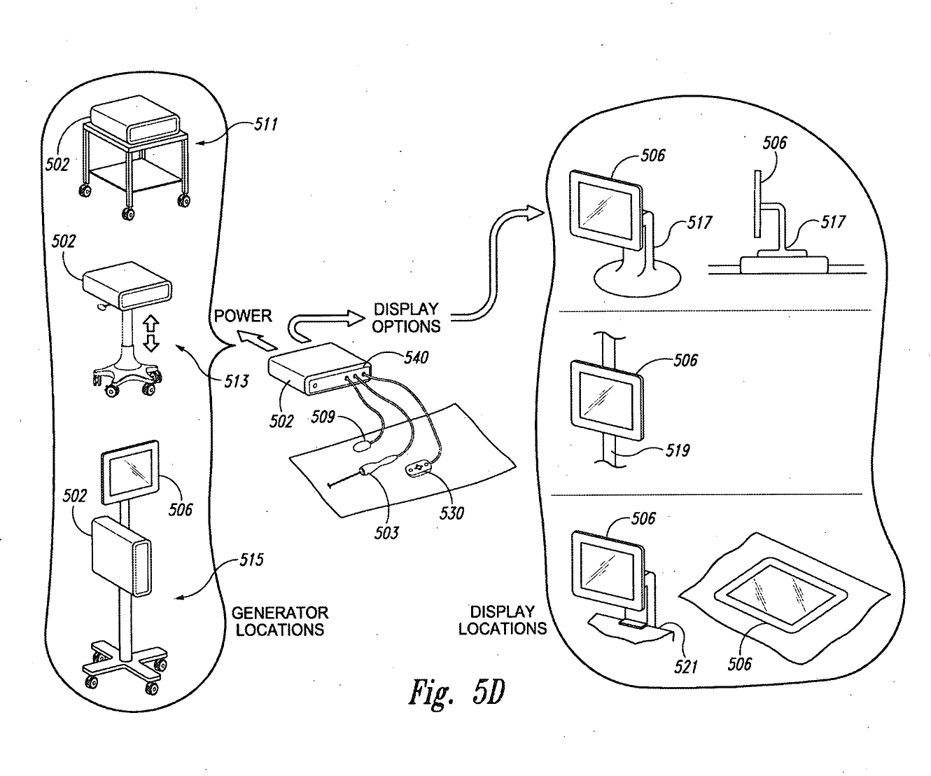

FIGS. 5A and 5B are isometric views of a generator system 500 (“system 500”) configured in accordance with yet another embodiment of the present technology, and FIG. 5C is a perspective view of the system 500 in a clinical setting. The system 500 includes features generally similar to the features of the systems 300 and 400 described above with reference to FIGS. 3A-4G. For example, the system 500 includes a generator assembly 502 and a display 506 mounted on a stand assembly 504. Similar to the system 400 of FIGS. 4A-4G, the generator assembly 502 and the display 506 are spaced apart from one another, and the generator assembly 502 can be detached from the stand assembly 504 for use as a standalone device.

The stand assembly 504 can include a maneuverable base portion 510 and a support member 516 extending therefrom to which the generator, assembly 502 is attached. The generator assembly 502 can be carried by the support member 516 in a lateral orientation (FIG. 5A), a longitudinal orientation (FIG. 5B), and/or any other suitable orientation. The support member 516 can also carry the display 506 and can be configured to lower the display 506 (FIG. 5A), raise the display 506 (FIG. 5B), and/or align the display 506 with other monitors 505 (FIG. 5C). The display 506 may be lowered, for example, to accommodate operators in a seated position and/or provide for compact storage, and may be raised to, for example, accommodate operators in a standing position.

FIG. 5D is a conceptual illustration of various modular configurations of the system 500 of FIGS. 5A and 5B. As shown in FIG. 5D, the generator assembly 502 can be positioned on a cart or table 511 or a dedicated stand 513 that can be adjusted per operator preference, or the generator assembly 502 can be mounted to a pole 515 (e.g., an I.V. pole) with the display 506 optionally mounted overhead. Similarly, the display 506 can also accommodate various configurations. For example, the display 506 can be mounted on or otherwise attached to a stand 517, a pole 519, a bed rail 521, and/or other suitable structures. In any of these configurations, the generator assembly 502 can be operably coupled to a neuromodulation device 503, a remote control device 530, a neutral or dispersive electrode 509, and/or other suitable peripheral devices via a cable interface 540 and provide energy (e.g., RF energy) for neuromodulation procedures.

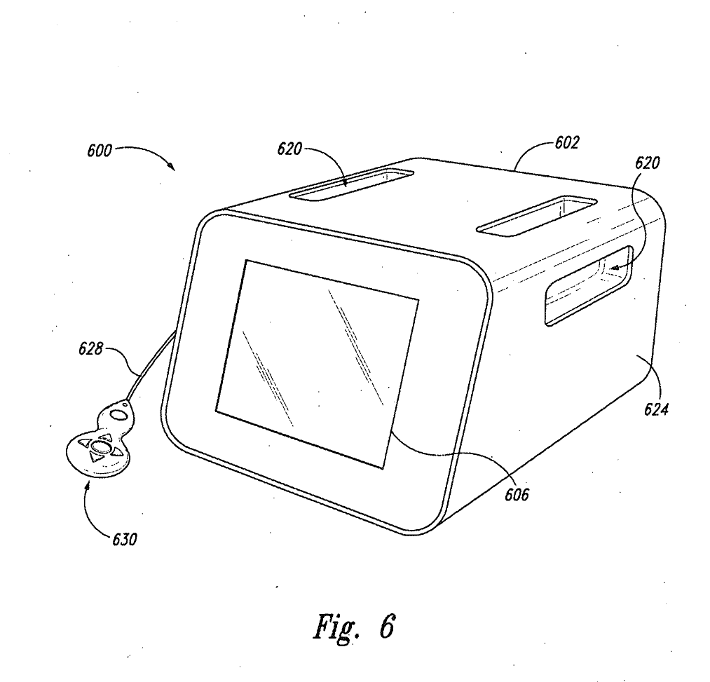

FIG. 6 is an isometric view of a generator system 600 (“system 600”) configured in accordance with a further embodiment of the present technology. The system 600 can include features generally similar to the features of the systems 300, 400 and 500 described above with reference to FIGS. 3A-5D. In this embodiment, however, the system 600 is a standalone console that integrates a generator assembly 602 and a display 606 into a single housing 624. As shown in FIG. 6, the system 600 can include handle portions 620 to facilitate transport and a remote control device 630 to initiate and control energy delivered by the generator assembly 602. The all-in-one system 600 may not require a stand assembly, and therefore may provide for substantially compact storage on a shelf or in a cabinet.

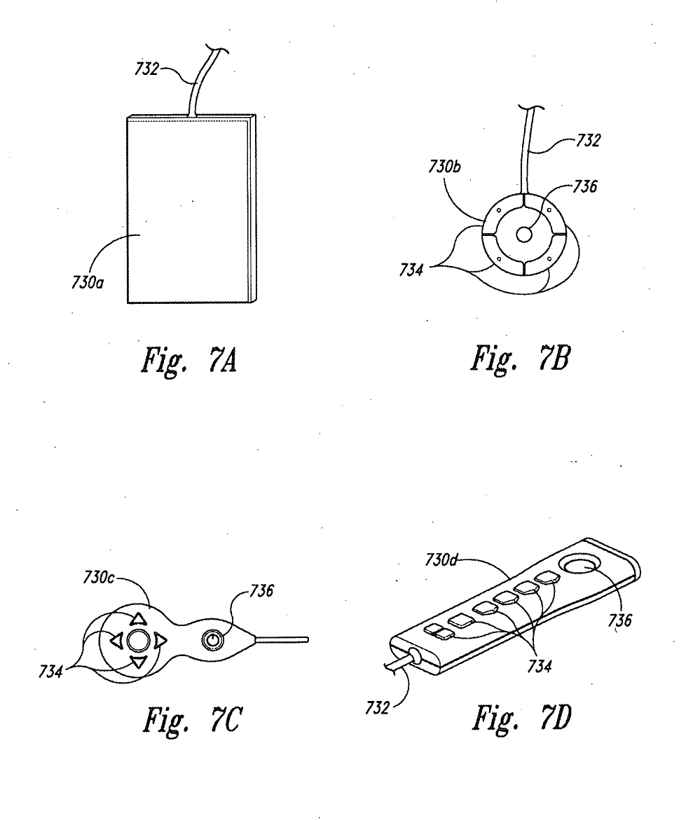

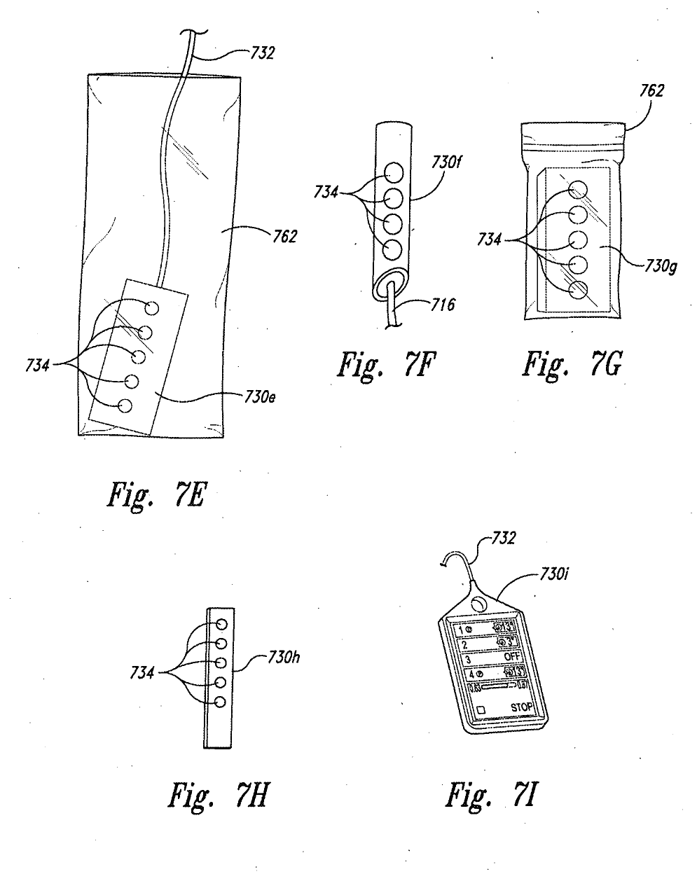

FIG. 7 illustrates a plurality of remote control devices 730 (identified individually as first through eighth remote control devices 730a-h, respectively) for use with generator systems configured in accordance with embodiments of the present technology, such as the generator systems 300-600 described above. The remote control devices 730 can be configured to be disposable, non-disposable, hardwired to a generator assembly (e.g., the generator assemblies 302, 402, 502 and 602 described above) via a cable 732, or wirelessly coupled to a generator assembly. For example, the first remote control device 730a is a foot pedal that can be pressed or otherwise manipulated to activate energy delivery. The first remote control device 730a can be pneumatically or electrically coupled to the generator assembly via the cable 732. The second and third remote control devices 730b and 730c are durable handheld devices that include a plurality of buttons for controlling energy delivery. The buttons can be oriented in a circular pattern and/or other easily identifiable button configuration that allows the operator to control energy delivery without having to look down at the remote control devices 730b, 730c. The fourth remote control device 730d is a disposable, hardwired controller that is shown enclosed in a bag 762 for use in a sterile field. The fifth remote control device 730e is configured to be mounted on the catheter of a neuromodulation device (e.g., the elongated shaft 116 shown in FIG. 1), and can include finger switches, buttons, and/or other actuators to control energy delivery. The fifth remote control device 730e can therefore be sterilized with the neuromodulation device and provide a convenient control means for the operator who may need to manipulate both the remote control device 730e and the neuromodulation device simultaneously or approximately simultaneously. The sixth and seventh remote control devices 730f and 730g are wireless devices, and may therefore increase the flexibility of the system. The sixth remote control device 730f, for example, is a durable device that is shown concealed in a sterile bag 762, whereas the seventh remote control device 730g is disposable and can be discarded after use. The eighth remote control device 730h includes features (e.g., a touchscreen) generally similar to the features of the remote control device 430 described above with reference to FIGS. 4A-4G. Although only some of the remote control devices 730 shown in FIG. 7 are enclosed in a sterile bag 762, it will be understood that any of the other remote control configurations can also be enclosed in such a bag for use in the sterile field. Additionally, a person skilled in the art will understand that the remote control devices 730 can have various other configurations for controlling energy delivery. For example, the features of one remote control device 730 shown in FIG. 7 can be combined with the features of another remote control device 730 and/or that some of the features of the remote control device 730 can be omitted. In other embodiments, the features of the remote control devices 730 described above can be integrated in a handle of a neuromodulation device (e.g., the handle assembly 134 shown in FIG. 1).

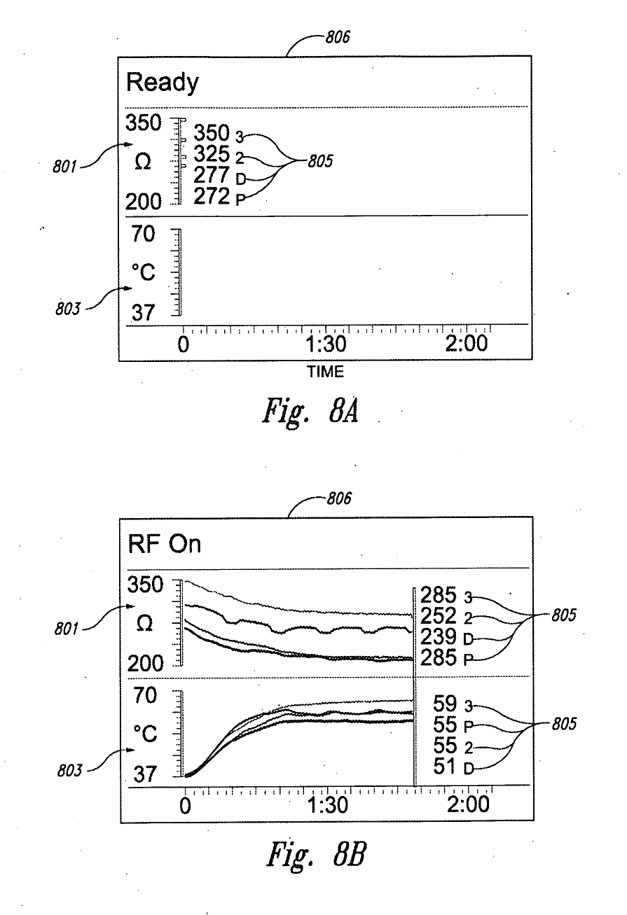

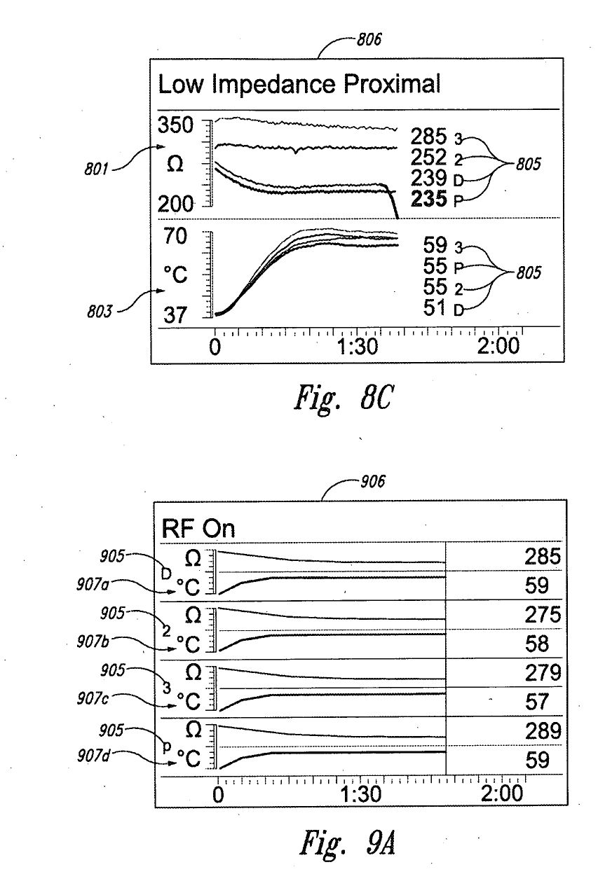

FIGS. 8A-11B are a series of screen shots illustrating various displays for generator systems configured in accordance with aspects of the present technology. The displays can be viewed on any of the displays 306-606 described above, on separate monitors or screens in a clinical setting, a touchscreen of a remote control device, and/or on other suitable devices. For example, FIGS. 8A-8C illustrate screen shots on a display 806 configured in accordance with an embodiment of the present technology. The display 806 includes an impedance vs. time graph used to plot the impedance of RF channels corresponding to one or more electrodes on a neuromodulation device and a temperature vs. time graph used to plot the temperature at each electrode during the treatment procedure. The graphs are updated in real time to provide the operator with feedback before, during, and after energy delivery.

In the embodiment illustrated in FIG. 8A, the impedance and temperature graphs include indicators that distinguish four RF channels (e.g., corresponding to four electrodes) from one another. The proximal electrode is identified as “P,” the distal electrode is identified as “D,” and the intermediate electrodes between the proximal and distal electrodes are identified as “2” and “3.” In other embodiments, the display 806 can include other indicators to distinguish the RF channels (e.g., different colors, symbols, etc.), and include more or fewer indicators depending upon the number of RF channels provided by the generator assembly.

During an initial stage of a neuromodulation procedure (i.e., before energy delivery), the display 806 can indicate the impedance of each RF channel according to magnitude. An operator can use this information to determine whether proper contact has been made by the electrodes at the target site. As further shown in FIG. 8A, the display 806 can also include a status indicator (e.g., “Ready” message) that communicates information to the operator regarding the status of the system.

FIG. 8B illustrates the display 806 during an energy application stage of the treatment procedure (e.g., as indicated by the “RF On” message on the display 806). The impedance and temperature of each RE channel can be plotted in real time as a separate curve on the impedance and temperature graphs, therefore allowing the operator to track the operating conditions of all displayed RF channels. As shown in FIG. 8C, when one of the electrodes goes outside of a predetermined impedance or temperature range, the display 806 can indicate the change to the user by displaying a warning message (e.g., “Low Impedance Proximal”) and/or highlight the change in the associated curve on the appropriate graph. In other embodiments, the display 806 may visually indicate different information and/or have a different arrangement.

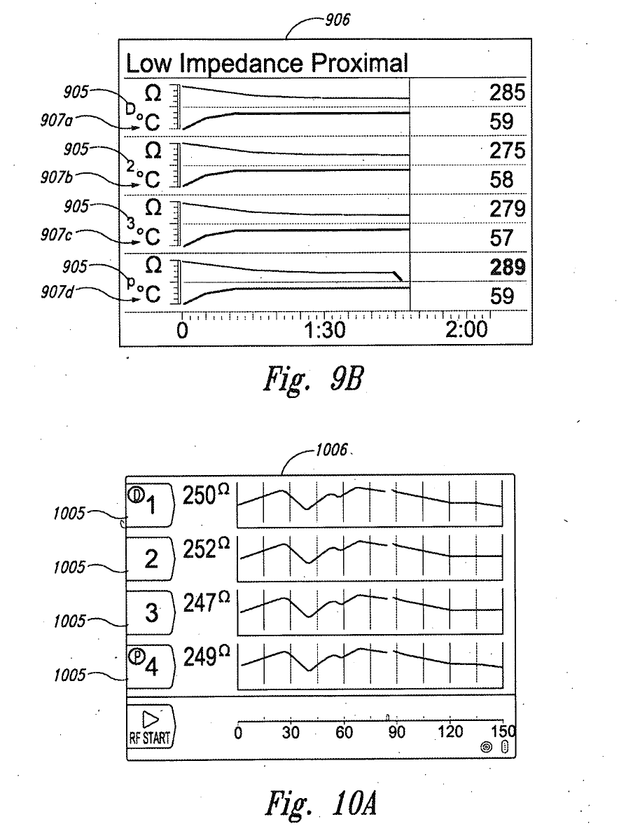

FIGS. 9A and 9B are screen shots illustrating a generator display 906 configured in accordance with other aspects of the present technology. The display 906 includes features generally similar to the features of the display 806 shown in FIGS. 8A-8C. For example, the display 906 includes impedance and temperature plots vs. time for each RF channel, and is configured to indicate when one of the RF channels is outside a predefined operating range (e.g., as shown in FIG. 9B). However, rather than grouping the impedance plots of all the RF channels together on a single impedance graph and the temperature plots of all the RF channels together on a single temperature graph, the display 906 of FIGS. 9A and 9B selectively groups the impedance and temperature plots for each RF channel. For example, referring to FIG. 9A, each RF channel (D, 2, 3, P) can have an individual impedance and temperature plot associated with it. In various embodiments, the display 906 can be configured to allow the operator to navigate between the RF channel-specific plots shown in FIGS. 9A and 9B and the parameter-specific plots shown in FIGS. 8A-8C. In further embodiments, the displays 806, 906 can include different and/or additional graphs associated with other operating parameters (e.g., power).

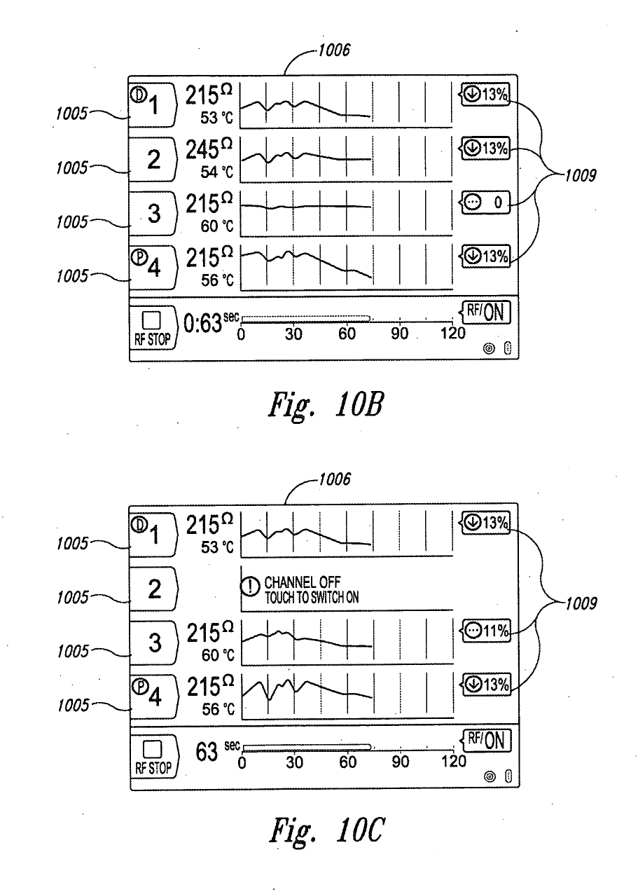

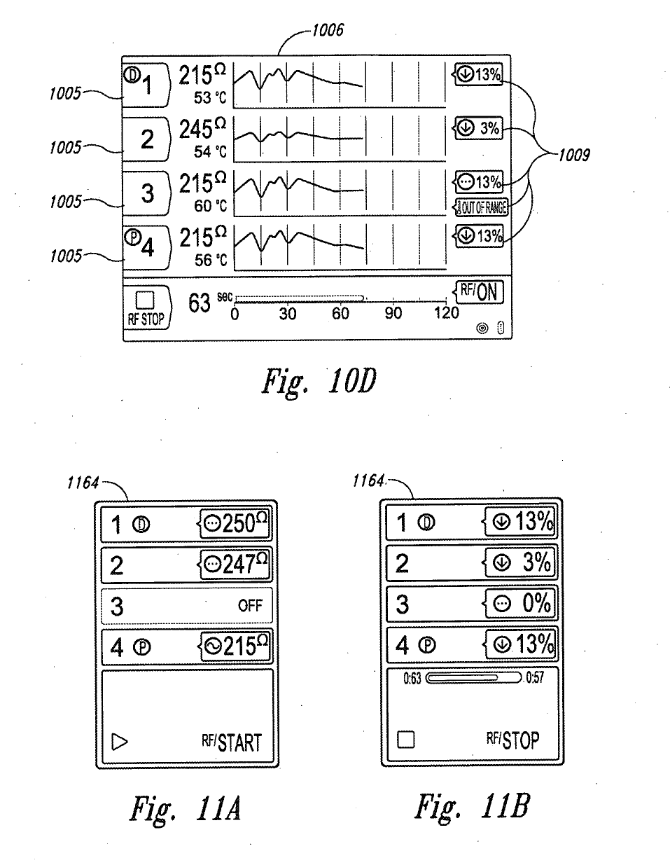

FIGS. 10A-10D are a series of screen shots illustrating a display 1006 configured in accordance with further aspects of the present technology. As shown in FIG. 10A, the display 1006 can include RF channel indicators (e.g., 1, 2, 3, 4 . . . n) corresponding to one or more electrodes coupled thereto. Before RF energy application, the display 1006 can provide a numerical impedance display for each RF channel and a graphical display of the impedance vs. time for each RF channel (e.g., over a two minute period) to show patterns and indicate whether good contact has been made and maintained at the treatment site. In various embodiments, the display 1006 can be a touchscreen and can accordingly include command buttons, such as an “RF START” button (FIG. 10A) that can be used to initiate RF energy delivery.

During RF energy delivery, the display 1006 can include a timer that indicates the elapsed time (FIG. 10B) and an “RF STOP” button that can be used to terminate energy delivery. The display 1006 can also include real time numerical displays of impedance and temperature measurements corresponding to each RF channel for quick reference checks and corresponding graphs that plot changes in impedance in real time. As further shown in FIG. 10B, the display 1006 can also include visual cues (e.g., down 13%, down 3%, etc.) that identify parameter change patterns for the operator. The display 1006 can further provide a clear signal regarding the state of each RF channel, such as when a channel is not turned on (FIG. 10C), and/or indicate when an RF channel is outside the predetermined operating conditions (FIG. 10D). For example, the display 1006 can provide a warning indicator that foreshadows when an RF channel is outside a predetermined temperature range before the channel is automatically switched off. In other embodiments, the display 1006 can include other and/or additional features, such as plots (e.g., temperature vs. time plots) associated with each RF channel.

FIGS. 11A and 11B are screen shots of a touchscreen 1164 on a remote control device (e.g., the remote control device 430 of FIGS. 4A-4G) configured in accordance with aspects of the present technology. The touchscreen 1164 can be coupled or synchronized to a larger display (e.g., the display 1006 of FIGS. 10A-10D) and provide information in an abbreviated format. As shown in FIG. 11A, before energy delivery the touchscreen 1164 can display the activation status and current impedance of each RF channel and provide visual signals as to whether or not the RF channel is stable. For example, if the generator system to which the touchscreen 1164 is coupled detects that the RF channel has not made sufficient contact at the target site, the touchscreen 1164 can provide a visual indicator to that effect (e.g., the symbol shown adjacent the numerical impedance value on RF channel 4). Once the system is sufficiently stable, the operator can begin RF energy delivery by pressing an “RF/START” button on the touchscreen 1164. During energy delivery, the touchscreen 1164 can display the procedure time (FIG. 11B) and indicate the changes in impedance of each RF channel. At any point during the procedure, the operator can press the “RF/STOP” button to terminate energy delivery.

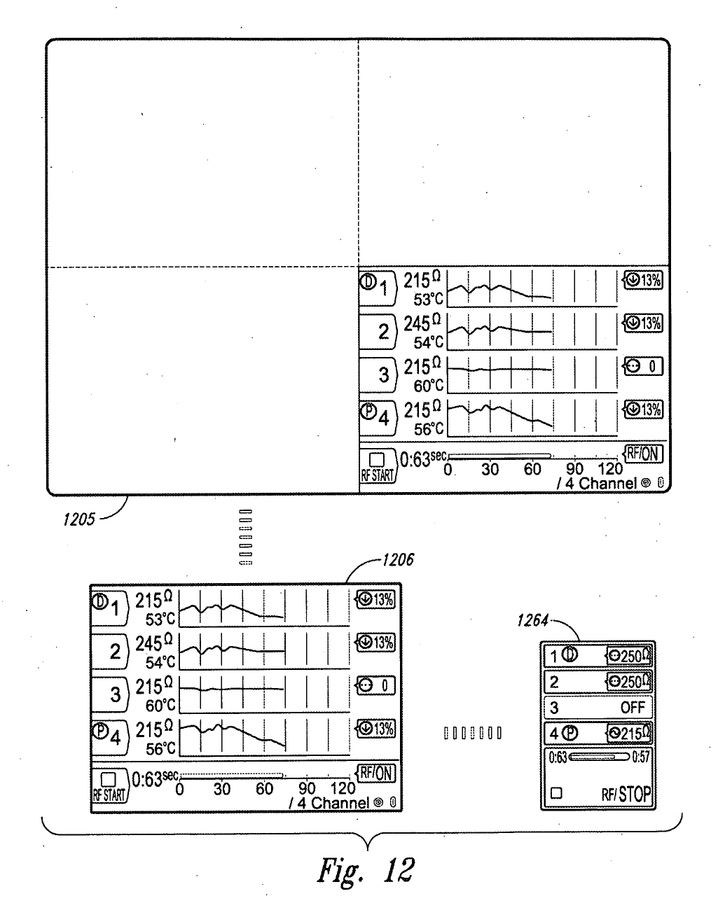

In various embodiments, various displays of a neuromodulation system can be integrated to provide various viewing options. For example, FIG. 12 illustrates the integration of a remote screen 1205, a display 1206 of a generator system, and a touchscreen 1264 of a remote control device in accordance with an embodiment of the present technology. The display 1206 can be replicated as a picture-in-picture inset on a portion of the larger screen 1205 (e.g., positioned in a clinical setting or in a remote lab) and the touchscreen 1264 can display condensed information from the display 1206. As such, the display system can provide a plurality of viewing options for the operator and other users to facilitate monitoring neuromodulation procedures.

### IV. Related Anatomy and Physiology

The Sympathetic Nervous System (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

1. The Sympathetic Chain

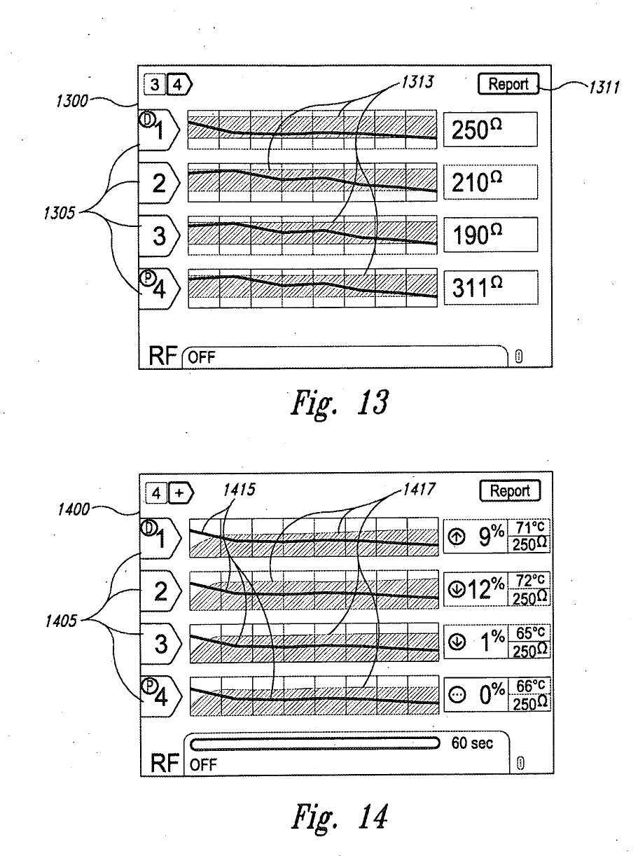

As shown in FIG. 13, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

2. Innervation of the Kidneys

As FIG. 14 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion; the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

3. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well known adverse consequences of chronic sympathetic over activity, such as hypertension, left ventricular hypertrophy, ventricular arrhythmias, sudden cardiac death, insulin resistance, diabetes, and metabolic syndrome.

### (i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na+) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

### (ii) Renal Sensory Afferent Nerve Activity

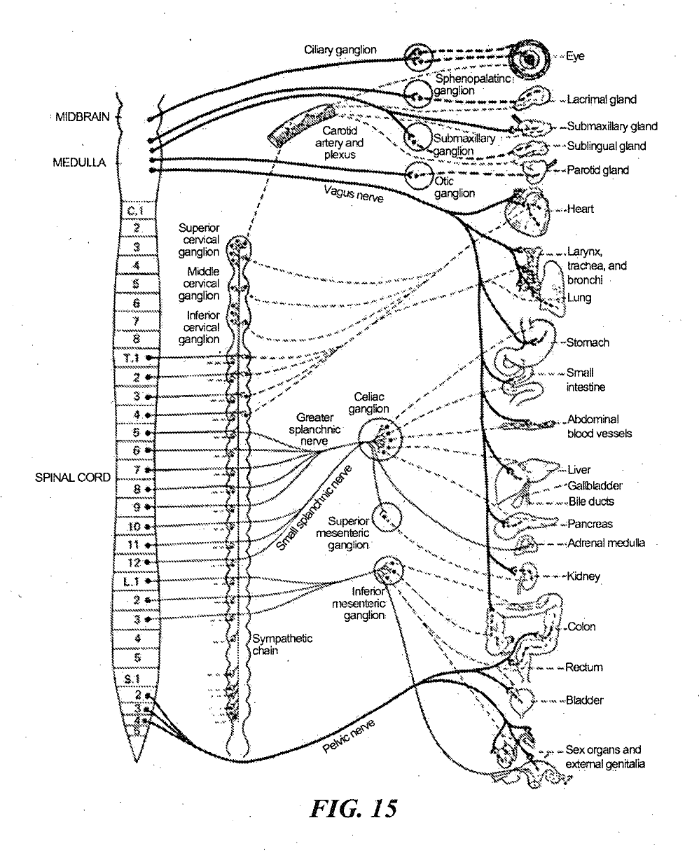

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 15A and 15B, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 13. For example, as previously discussed, a reduction in central sympathetic drive may reduce the insulin resistance that afflicts people with metabolic syndrome and Type II diabetics. Additionally, patients with osteoporosis arc also sympathetically activated and might also benefit from the down regulation of sympathetic drive that accompanies renal denervation.

C. Achieving Intravascular Access to the Renal Artery

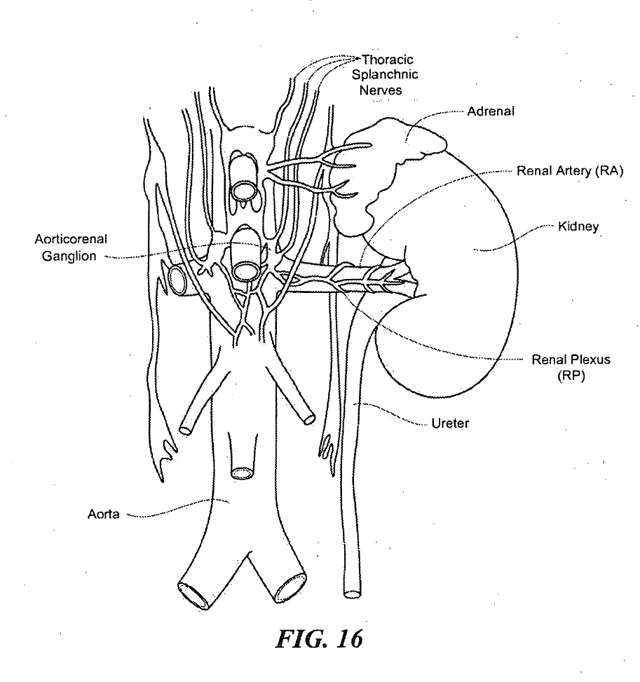

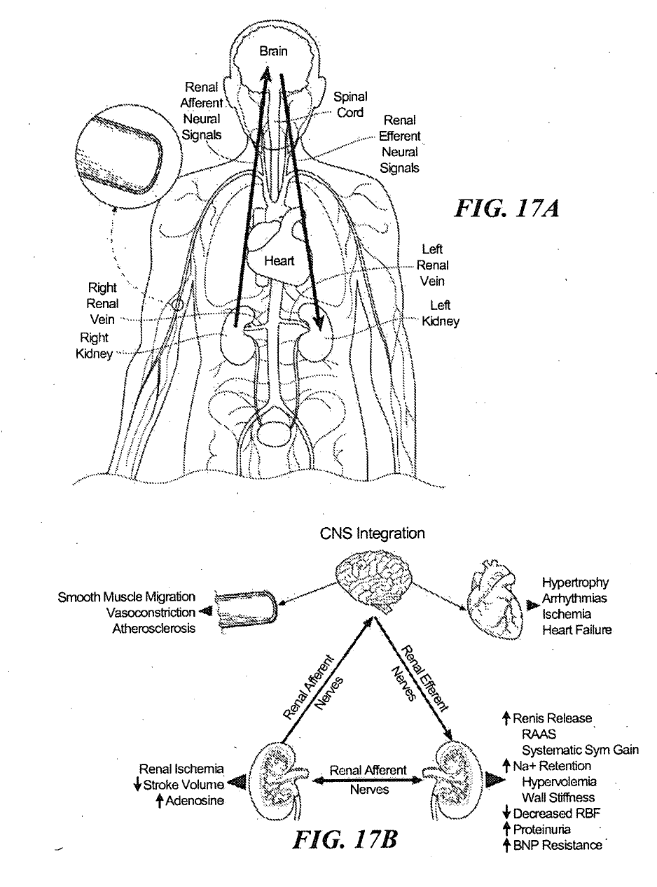

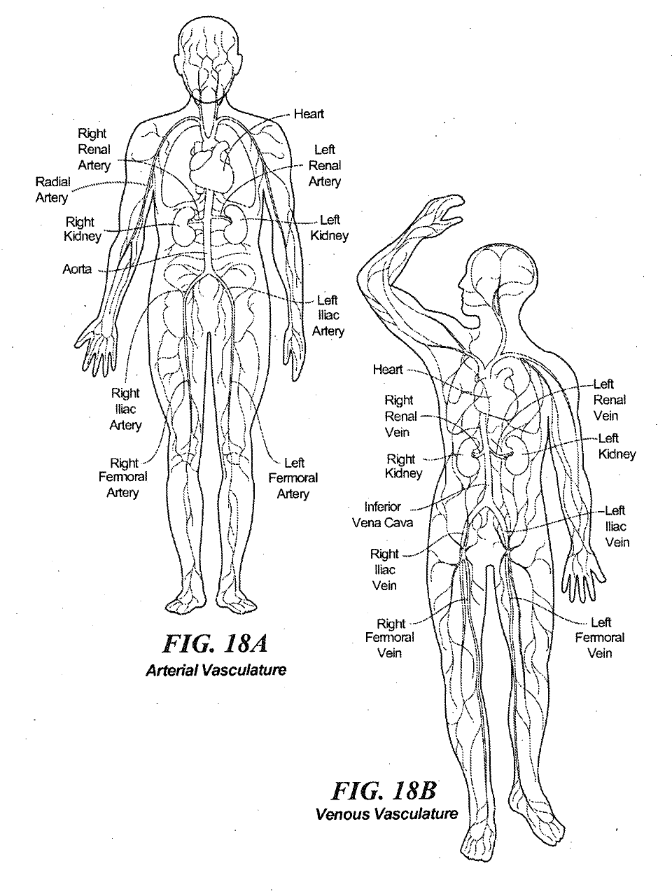

In accordance with the present technology, neuromodulation of a left and/or right renal plexus (RP), which is intimately associated with a left and/or right renal artery, may be achieved through intravascular access. As FIG. 16A shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 16B shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch; down the descending aorta and into the renal arteries using standard angiographic technique.

D. Properties and Characteristics of the Renal Vasculature

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e. cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its' adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, a full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility: and (f) as the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta induced by respiration and/or blood flow pulsatility. A patient's kidney, which is located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### V. Examples

1. A generator system for neuromodulation therapies, the generator system comprising:
a stand assembly;
a generator assembly carried by the stand assembly and having at least one port configured to operably couple the generator assembly to a neuromodulation device, wherein the generator assembly is configured to provide radio frequency (RF) energy to the neuromodulation device;
a display operably coupled to the generator assembly, the display being configured to indicate operating conditions of the generator system during energy delivery; and
a user interface operably coupled to the generator assembly and configured to activate and/or modulate the RF energy.

2. The generator system of example 1 wherein the generator assembly is removably attached to the stand assembly, the generator assembly being configured to provide RF energy to the neuromodulation device independent of the stand assembly.

3. The generator system of example 2 wherein:
the generator assembly is configured to be mounted to a permanent structure when the generator assembly is separated from the stand assembly; and
the generator system further comprises a cable interface module spaced apart from and operably coupled to the generator assembly, the cable interface module being configured to operably couple the generator assembly to the neuromodulation device.

4. The generator system of example 1 wherein:
the stand assembly comprises a base portion and a body portion;
the display is integrated with the generator assembly to form an integrated console; and
the console is configured to be removably supported by the body portion of the stand assembly.

5. The generator system of example 1 wherein:
the at least one port of the generator assembly is a first port;
the stand assembly comprises an enclosure configured to receive the generator assembly and a second port configured to operably couple the generator assembly to the neuromodulation device, the second port being spaced apart from the generator assembly; and
the display is spaced apart from the generator assembly.

6. The generator system of example 1 wherein the stand assembly comprises a height adjustable support configured to carry the display.

7. The generator system of example 1 wherein the generator assembly is operably coupled to a remote display, the remote display being configured to visually indicate the operating conditions during neuromodulation.

8. The generator system of example 7 wherein the generator assembly is removable from the stand assembly, and wherein the generator assembly is wirelessly coupled to the remote display.

9. The generator system of example 1 wherein:
the user interface is a remote control device; and
at least one of the stand assembly and the generator assembly include a recessed portion configured to removably retain the remote control device.

10. The generator system of example 1 wherein the user interface comprises a foot pedal.

11. The generator system of example 1 wherein the user interface comprises a remote control device that is wirelessly coupled to the generator assembly.

12. The generator system of example 1 wherein the user interface comprises a disposable remote control device.

13. The generator system of example 1 wherein the user interface comprises a remote control device coupled with a proximal portion of the neuromodulation device.

14. The generator system of example 1 wherein the user interface comprises a remote control device having a touchscreen, and wherein the touchscreen is configured to display one or more operating conditions during neuromodulation.

15. The generator system of example 1 wherein:
the user interface comprises a remote control device; and
the generator system further comprises a bag configured to form a sterile barrier around at least a portion of the remote control device.

16. The generator system of example 1 wherein the generator assembly comprises a plurality of RF channels that operably connect with one or more electrodes on the neuromodulation device.

17. The generator system of example 16 wherein the display is configured to visually indicate operating conditions of the individual RF channels, wherein the operating conditions include at least one of impedance and temperature.

18. The generator system of example 16 wherein the display is configured to visually indicate when one of the operating conditions is outside of a predetermined temperature or impedance range.

19. The generator system of example 16 wherein the display is configured to graphically illustrate at least one of impedance and temperature of each RF channel.

20. The generator system of example 1 wherein:
the generator assembly is operably coupled to a remote monitor;
the user interface comprises a remote controller unit having a touchscreen; and
the display, remote monitor, and the touchscreen are configured to illustrate various operating conditions in varying levels of specificity, the touchscreen being configured to illustrate fewer details of the operating parameters than the display and the remote monitor.

21. A neuromodulation system, comprising:
a treatment device including
   an elongated shaft having a distal portion and a proximal portion; and
   a therapeutic assembly at the distal portion,
   wherein the therapeutic assembly is configured to purposefully apply energy to a target treatment site within a human patient;
means for generating energy for the therapeutic assembly of the treatment device;
a display configured to illustrate operating conditions during energy application, the operating conditions including at least one of temperature and impedance; and
a remote control device configured to control energy delivery to the treatment device.

22. The neuromodulation system of example 21, further comprising a stand assembly having a body portion configured to removably receive the means for generating energy for the therapeutic assembly of the treatment device.

23. The neuromodulation system of example 21 wherein:
the therapeutic assembly, comprises at least four electrodes;
the means for generating energy comprises a radio frequency (RF) generator configured to provide RF channels corresponding to the individual electrodes of the therapeutic assembly; and
the display is configured to visually indicate the individual operating conditions of the RF channels.

24. The neuromodulation system of example 21 wherein:
the treatment device comprises a handle assembly at the proximal portion of the elongated shaft; and
the remote control device is at least proximate to the handle assembly of the treatment device.

25. The neuromodulation system of example 21 wherein:
the treatment site is at least proximate to a renal artery; and
the therapeutic assembly is configured to purposefully apply energy to the renal artery to modulate neural fibers that innervate a kidney.

26. The neuromodulation system of example 21 wherein the means for generating energy and the display form an integrated console.

27. The neuromodulation system of example 21 wherein the means for generating energy and the display are separate components spaced apart from one another on the stand assembly.

28. The neuromodulation system of example 21, further comprising a remote display configured to display operating conditions during neuromodulation.

29. The neuromodulation system of example 21 wherein the remote control device comprises at least one of a foot pedal, a touchscreen, a wireless remote control device, and a disposable remote control device.

30. A method of providing therapeutic neuromodulation, the method comprising:
delivering a therapeutic assembly at a distal portion of a treatment device at least proximate to a renal artery, the therapeutic assembly having a plurality of electrodes;
delivering individual channels of RF energy from a generator assembly to the individual electrodes, the generator assembly being operably coupled to a proximal portion of the treatment device; and
displaying operating conditions of the individual RF channels on a display, wherein the operating conditions include at least one of temperature and impedance.

31. The method of example 30, further comprising controlling the delivery of the RF energy via a remote control device operably coupled to the generator assembly.

32. The method of example 30 wherein displaying operating conditions comprises displaying an impedance plot and a temperature plot of each RF channel in real time during neuromodulation.

33. The method of example 32, further comprising visually indicating to a user when one of the electrodes falls outside a predetermined impedance range and/or a predetermined temperature range.

34. The method of example 30 wherein displaying operating conditions comprises displaying visual cues associated with an activation state and/or operating status of each electrode.

35. The method of example 30 wherein displaying operating conditions comprises:
associating each electrode with a different indicator; and
plotting the impedance and/or temperature of each electrode during neuromodulation on a graph using the different indicators.

36. The method of example 30, further comprising:
controlling the delivery of the RF energy from the generator assembly to the individual electrodes via a remote control device operably coupled to the generator assembly; and
displaying on the remote control device visual indicators associated with an operating status of each electrode.

37. The method of example 30, further comprising supporting the generator assembly and the display on a height adjustable stand assembly.

### VI. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A neuromodulation system, as substantially disclosed herein.

2. A generator assembly for a neuromodulation system, as substantially disclosed herein.

3. A method of treating a patient, as substantially disclosed herein.

4. A method of performing neuromodulation, as substantially disclosed herein.
